(19) United States
(12) Patent Application Publication
(10) Pub. No.: US 2013/0045318 A1
(43) Pub. Date: Feb. 21, 2013

(54) SALTY TASTE ENHANCER AND KELP EXTRACT COMPRISING THE SAME

(71) Applicants: Akihiko Watanabe, Kanagawa (JP); Azusa Nakatoh, Kanagawa (JP); Takeshi Oikawa, Kanagawa (JP)

(72) Inventors: Akihiko Watanabe, Kanagawa (JP); Azusa Nakatoh, Kanagawa (JP); Takeshi Oikawa, Kanagawa (JP)

(73) Assignee: TAKASAGO INTERNATIONAL CORPORATION, Tokyo (JP)

(21) Appl. No.: 13/580,016

(22) PCT Filed: Apr. 8, 2011
(86) PCT No.: PCT/JP2011/059331

§ 371 Date: Aug. 20, 2012

(30) Foreign Application Priority Data
Apr. 8, 2010 (JP) 2010-089332
Apr. 4, 2011 (JP) 2011-082883

(51) Int. Cl. A23L 1/221; C07C 45/82; C12S 3/00; A23L 2/56; C07C 29/80; C07C 47/21; C07C 49/203; C07C 49/21; C07C 47/02; C07C 33/025

(52) U.S. Cl. 426/590; 568/448; 568/492; 435/267; 568/909.5; 568/417; 568/410; 568/377; 426/650; 426/492

(57) ABSTRACT

To provide a salty taste enhancer which exerts a flavor enhancing effect comparable to sodium chloride without imparting any undesirable flavor such as harsh taste or odd smell, a method for producing the same, a kelp extract comprising the salty taste enhancer, and a food or drink having enhanced salty taste and flavor which comprises the salty taste enhancer or the kelp extract. A salty taste enhancer which comprises a volatile component with a molecular weight of less than 200 derived from a kelp.

### TECHNICAL FIELD

The present invention relates to a salty taste enhancer capable of enhancing salty taste of foods and a method for producing the salty taste enhancer. More specifically, it relates to a salty taste enhancer capable of enhancing salty taste of foods or drinks over a wide range with the use of volatile components derived from kelp, a method for producing the salty taste enhancer, a kelp extract comprising the salty taste enhancer, and a food or drink comprising the salty taste enhancer or the kelp extract.

### BACKGROUND ART

Although sodium chloride is a mineral that is essentially required for human life, it has been recently reported that the excessive intake of sodium chloride induces various life style-related diseases, for example, hypertension, arteriosclerosis and various life style-related diseases induced thereby, stomach cancer and the like. In addition to the use as an essential nutrient, sodium chloride has been widely used for preserving and seasoning foods over a long time. Moreover, sodium chloride exerts important effects of improving the tastes of foods. These effects include not only the impartation of salty taste but also the enhancement of flavors. For example, it has been known that sodium chloride has an effect of enhancing flavors. Owing to this effect, a processed food containing a large amount of sodium chloride can give high satisfaction, which such a satisfaction is not obtained by taking a low-salt food. In addition to the entire thickness and sharpness, the top impact-enhancing effect achieved by sodium chloride that is used in an appropriate amount is highly important in seasoning as a secondary effect of sodium chloride. Under these circumstances, there have been required various substitutes for sodium chloride and salty taste enhancers which ensure the preparation of delicious dishes while reducing sodium chloride content without damaging the tastes of foods.

To meet these requirements, various substitutes for sodium chloride have been reported hitherto. For example, inorganic salts typified by potassium chloride have been widely used as substitutes for sodium chloride. However, it is known that these inorganic salts have odd tastes such as characteristic harshness. To overcome this problem, for example, Patent Literature 1 discloses to regulate the inherent odd taste of potassium chloride by using potassium chloride together with a kelp extract and trehalose, and Patent Literature 2 discloses a salty seasoning having a low sodium content which comprises sodium chloride, potassium chloride, a kelp extract and flavoring components at a definite ratio. Although these substitutes have salty taste due to potassium chloride contained therein, a common kelp extract is employed in both cases. Therefore, these products are not so widely usable because of the characteristic kelp flavor such as the smell of the sea. In addition, the flavor enhancing effect of sodium chloride cannot be sufficiently established thereby.

Also, various salty taste enhancers have been developed so far. For example, Patent Literature 3 discloses a method for enhancing salty taste using a saturated aliphatic monocarboxylic acid, and Patent Literature 4 discloses a method for enhancing salty taste by using spilanthol with an extract of a plant belonging to the genus Allium such as shallot or onion. However, both products cannot achieve a sufficient salty taste enhancing effect and are not widely usable. Furthermore, the flavor enhancing effect of sodium chloride cannot be sufficiently established thereby.

On the other hand, various attempts have been made hitherto to obtain concentrates or extracts of marine products, such as fishes and marine algae, which have a flavor or taste closely similar to the inherent flavor or taste of the marine products. For example, Patent Literature 5 and Patent Literature 6 disclose a method for producing a concentrated extract of a marine product. This method comprises: adding water to the raw materials of the marine product, for example, fishes, marine algae, shellfishes or crustaceans, and then, for example vacuum- or steam-distilling the same and concentrating flavor components thus obtained to give a flavor component; extracting the distillation residue and concentrating the same to give a taste component; and mixing the flavor component with the taste component thus obtained to give a concentrated extract of the marine product having an excellent flavor. Patent Literature 7 and Patent Literature 8 discloses a method for reducing the fishy smell characteristic to marine algae by extracting a marine alga such as kelp with an alcoholic solvent at a low temperature and then treating the marine alga extract with a synthetic adsorbent. However, these concentrated marine product extracts are produced to utilize the inherent flavors of the marine products. Therefore, it has never been known that the flavor components thus obtained have a salty taste-enhancing effect or a flavor enhancing effect exhibited by sodium chloride.

Furthermore, Patent Literature 9 discloses a method for extracting flavor components from kelp, which comprises soaking a kelp powder in an aqueous alcohol, and then washing the aqueous alcohol containing flavor components with carbon dioxide having been liquefied or being in a subcritical or supercritical state and collecting the flavor components, and a method for producing a seasoning which comprises the flavor components thus obtained. However, the purpose of these methods is to enhance the flavor inherent to kelp, such as the smell of the sea, and, therefore, the obtained product is not suitable as a widely usable salty taste enhancer without a need for the kelp flavor. Moreover, it is never disclosed therein that the obtained flavor components have a salty taste-enhancing effect or a flavor enhancing effect exhibited by sodium chloride.

### CITATION LIST

### Patent Literature

PTL 1 JP-A-2009-65978
PTL 2 JP-A-6-7111
PTL 3 JP-A-5-184326
PTL 4 JP-A-2006-296357
PTL 5 JP-A-9-9908
PTL 6 JP-A-2004-89141
PTL 7 JP-A-2003-144102
PTL 8 JP-A-2007-37475
PTL 9 JP-A-2001-78705

### SUMMARY OF INVENTION

### Technical Problem

The invention provides a salty taste enhancer, which is free from the various problems encountering in the existing substitutes for sodium chloride or salty taste enhancers or in which these problems have been overcome, a method for producing the same, a kelp extract having a salty taste-enhancing effect, and a food or drink comprising the salty taste enhancer or the kelp extract.

Accordingly, an embodiment of the invention is to provide a salty taste enhancer, which exerts a flavor enhancing effect comparable to sodium chloride without imparting any undesirable flavor such as harsh taste or odd smell, and a method for producing the same.

Another embodiment of the invention is to provide a kelp extract comprising the salty taste enhancer, and a food or drink which comprises the salty taste enhancer or the kelp extract and has enhanced salty taste and flavor.

### Solution to Problem

To solve the aforesaid problems, the present inventors conducted intensive studies and, as a result, found that volatile components of kelp have a salty taste-enhancing effect. The invention has been completed based on this finding.

Accordingly, the invention encompasses the following embodiments.

(1) A salty taste enhancer comprising a volatile component with a molecular weight of less than 200 derived from a kelp.

(2) The salty taste enhancer according to (1), wherein the kelp is an enzymatically treated kelp.

(3) The salty taste enhancer according to (1) or (2), which comprises a volatile component with a molecular weight of 200 or more derived from the kelp in an amount of less than 60% by weight relative to an amount of a volatile component with a molecular weight of 200 or more contained in the starting kelp.

(4) A method for producing the salty taste enhancer according to (1), which comprises distilling a kelp to give a salty taste enhancer comprising a volatile component with a molecular weight of less than 200 derived from the kelp.

(5) A method for producing the salty taste enhancer according to (3), which comprises distilling a kelp to give a salty taste enhancer comprising: a volatile component with a molecular weight of less than 200 derived from the kelp; and a volatile component with a molecular weight of 200 or more derived from the kelp in an amount of less than 60% by weight relative to an amount of a volatile component with a molecular weight of 200 or more which is contained in the starting kelp.

(6) The method according to (5), wherein a volatile component is captured from the start of the distillation to a point at which the volatile component is obtained in an amount of 20 ppm or less relative to the weight of the starting kelp.

(7) The method according to any of (4) to (6), wherein the distillation is conducted after the kelp was treated with an enzyme.

(8) A kelp extract which comprises a mixture of the salty taste enhancer according to any of (1) to (3) and an extract containing a non-volatile component of the kelp.

(9) The kelp extract according to (8), wherein the extract containing a non-volatile component of the kelp is an extract containing a non-volatile component of the kelp, which is obtained after distilling the kelp to remove a volatile component from the kelp.

(10) A food or drink comprising the salty taste enhancer according to any of (1) to (3).

(11) A food or drink comprising the kelp extract according to (8) or (9).

### Advantageous Effects of Invention

The salty taste enhancer according to the invention can enhance salty taste without giving any odd-after tastes such as harshness as shown by potassium chloride, exert a flavor enhancing effect, which is another effect of sodium chloride in foods or drinks, on foods or drinks, and impart top impact, which is lost by salt reduction, to a food or drink.

In the salty taste enhancer according to the invention, furthermore, the characteristic flavors (the smell of the sea, algal smell and threadiness) inherent to kelp are relieved. Therefore, it can exert the salty taste-enhancing effect and flavor enhancing effect on foods or drinks over a wide range without giving any unpleasant feeling.

### DESCRIPTION OF EMBODIMENTS

Next, the invention will be described in greater detail.

As described above, the salty taste enhancer according to the invention comprises a volatile component with a molecular weight of less than 200 derived from kelp. The salty taste enhancer according to the invention, which comprises a volatile component with a molecular weight of less than 200 derived from kelp, can be obtained by an arbitrary method for obtaining volatile components derived from kelp. Examples of the method include distillation of the starting kelp in the presence of a solvent, extraction of the starting kelp with a solvent, supercritical extraction thereof and the like. Among these methods, distillation is preferred.

The starting kelp to be used in the above method is not specifically restricted in production area, variety or the like, as long as it is an arbitrary dry kelp commonly used for preparing kelp broth. Typical examples of Japanese kelp varieties include Laminaria ochotensis, Saccharina japonica, Laminaria angustata, Laminaria diabolica and the like. Also, use can be made of Chinese kelp varieties. Either one kind of kelp or two or more kinds thereof may be used.

Next, a general method for obtaining the volatile components according to the invention by distillation will be illustrated. The starting kelp and a solvent are fed into, for example, a tightly closed container and heated. Then, the vapor and gas thus generated are captured in vacuo or by using a device, for example, a condenser tube. Thus, a solution containing volatile components derived from kelp can be obtained. In the invention, this solution can be directly used as a salty taste enhancer-containing solution. Alternatively, the volatile components derived from kelp may be separated from the solution by extracting or the like and, if needed, further purified to give a salty taste enhancer according to the invention.

To speak in greater detail, the starting kelp and the solvent are first introduced into a heating container equipped with a condenser tube, for example, a heating/stirring kettle equipped with a condenser tube. Next, the starting kelp and solvent are heated to the boiling point of the solvent or a temperature close thereto and then maintained in this state for a definite period of time. The vapor generated during this period is captured as a solution through the condenser tube. Examples of the solvent usable therein include water, a dilute aqueous ethanol solution, ethanol, methanol, acetone, diethyl ether, pentane, hexane, ethyl acetate and the like. Among these solvents, water is preferred. Although the starting kelp and solvent may be used at an arbitrary ratio, it is generally preferable, from the stand point of the efficiency in stirring or extracting, to control the weight ratio of the kelp: the solvent to 1:100 to 1:5 (more preferably 1:50 to 1:10).

The solvent and the volatile components in the kelp that which are evaporated by heating are cooled with the condenser tube and then captured and collected, in the form of a solution containing the volatile components in the kelp, in a collection container. The captured solution may be collected into the collection container by an arbitrary method. For example, the captured solution may be collected in fractions per unit time or unit amount. Alternatively, the whole captured solution may be collected at once. With respect to the capture time, it is preferred to continue the capture until all of the volatile components in the starting kelp are removed from the kelp solution. When the capture time is too long, however, the solvent such as water in the kettle is excessively reduced, which hinders the stirring. As a result, there arise some troubles such as an odd smell generated by scorched starting kelp. The heating time and the collection amount of the discharged solution also vary depending on the amount and kind of the starting kelp and the solvent employed, the size of the heating container, the capability of the condenser tube, the heating temperature and the like. In the case of using water as the solvent, the heating time may generally range from about 1 to 3 hours, though the invention is not restricted to this range. The heating temperature is preferably between the boiling temperature of the solvent and a temperature lower by 10° C. than the boiling temperature. The total amount of the captured solution is usually from about 20 to 40% by weight relative to the amount of the solvent employed as a starting material. In the case of collecting the solution in fractions, the amount of each fraction may be appropriately determined. For example, a capture container may be replaced each time the amount of the captured solution attains to a definite level, e.g., about 1 to 20% by weight relative to the amount of the starting solvent.

By setting a shorter capture time per fraction (i.e., unit capture time) or a smaller amount of the solution captured per fraction (i.e., unit capture amount) in the aforesaid fractional collection, the component distribution of the volatile components in each fraction can be narrowed. When the solution containing the kelp soaked therein is heated and thus a vapor is generated, a component having a smaller molecular weight vaporizes earlier with vaporization of the solvent. Therefore, the solution captured first contains low-molecular weight components in a large amount. The amount of the low-molecular weight components decreases while the ratio of high-molecular weight components increases each time replacing the capture container.

As the heating container to be used for the distillation in the invention, use can be made of any kind of container so long as a condenser tube can be attached thereto and the vapor discharged therefrom during the distillation can be collected through the condenser tube. Examples of such a container include a steam-type stirring kettle, an autoclave extraction kettle, a steam-jacketed kneader and the like. Although such a heating container is not restricted in material, it preferably has a stirring unit. Also, it is necessary that the heating container can be completely tightly closed excluding the section to which the condenser tube is attached. As the condenser tube, use can be made of, for example, a ground glass joint condenser (manufactured by IWAKI), and the like. Since it is important in the invention to capture low-molecular weight volatile components, the heating container and the condenser tube should be tightly closed so that these components can be surely captured. The expression “tightly closed” as used herein indicates a state wherein no part, other than the outlet of the condenser, contacts with external air.

As the results of studies by the inventors, it has been clarified that, among the volatile components derived from kelp, low-molecular weight volatile components exert a higher salty taste-enhancing capability. In the volatile components derived from kelp, high-molecular weight components contain components relating to the undesirable kelp smells such as the smell of the sea and algal smell. When the salty taste enhancer of the invention contains a large amount of the high-molecular weight volatile components, therefore, the salty taste-enhancing effect of the salty taste enhancer is deteriorated and, moreover, the characteristic flavors (the smell of the sea, algal smell and threadiness) inherent to kelp become obvious, which brings about undesirable results such as a reduction in preference and a narrowing in the application range of the salty taste enhancer. In the case of requiring a salty taste enhancer having a wide applicability and a high salty taste-enhancing capability, it is preferred to use the low-molecular weight fraction(s) of the volatile components derived from kelp. As the results of various tests and studies, it has been revealed that the salty taste enhancer of the invention preferably contains, as the active ingredient, volatile components with a molecular weight of less than 200, more preferably less than 170 and still more preferably less than 140. The salty taste enhancer of the invention preferably contains, as the active ingredient, volatile components with a molecular weight of 40 or more.

Supposing that the entire volatile components in the starting kelp have been collected, it is preferable that, in the salty taste enhancer of the invention, the amount of volatile components with a molecular weight of 200 or more derived from the starting kelp is controlled to less than 60% by weight, more preferably 0 or more and less than 50% by weight and still more preferably 0 or more and less than 40% by weight, relative to the total amount of the volatile components with a molecular weight of 200 or more that are contained in the starting kelp. It is also preferred that the amount of volatile components with a molecular weight of 170 or more and less than 200 derived from kelp is controlled to less than 80% by weight, more preferably 0 or more and less than 60% by weight and still more preferably 0 or more and less than 50% by weight, relative to the total amount of the volatile components with a molecular weight of 170 or more and less than 200 that are contained in the total volatile components derived from kelp. Furthermore, it is preferred that the amount of volatile components with a molecular weight of 140 or more and less than 170 derived from kelp is controlled to less than 75% by weight, more preferably 0 or more and less than 70% by weight, relative to the total amount of the volatile components with a molecular weight of less than 140 to 170 that are contained in the total volatile components derived from kelp.

To obtain a salty taste enhancer in such a preferred embodiment as described above (for example, a salty taste enhancer containing, as the active ingredient, the volatile components with a molecular weight of less than 200 derived from kelp), it is therefore preferred to divide the captured volatile components into a low-molecular weight fraction, which has a strong salty taste-enhancing effect and, therefore, fits for the purpose of enhancing salty taste, and a high-molecular weight fraction which has a weak salty taste-enhancing effect and, therefore, does not fit for the purpose of enhancing salty taste. This can be achieved by, for example, a method of treating the captured volatile components again by using a multiplate distillation column, or a method of, in the course of distilling the volatile components, fractionally capturing the volatile components to give a first half, i.e., a fraction containing low-molecular weight volatile components, and a second half, i.e., a fraction containing high-molecular weight volatile components. It is preferable to obtain a salty taste enhancer which contains, as the active ingredient, the low-molecular weight volatile components with a molecular weight of less than 200, preferably less than 170 and more preferably less than 140, which is derived from kelp, by using one or more of these methods.

More specifically speaking, for example, in the case of obtaining the volatile components with a molecular weight of less than 200 derived from kelp by distillation, the desired components can be obtained by collecting the solution captured from the start of the distillation of the solution containing the kelp soaked therein to the point at which the volatile components is obtained in an amount of 20 ppm or less (preferably 0.1 ppm or more and 20 ppm or less) relative to the weight of the starting kelp.

Also, in the case of obtaining the volatile components with a molecular weight of less than 170 derived from kelp by distillation, the desired components can be obtained by collecting the solution captured from the start of the distillation of the solution containing the kelp soaked therein to the point at which the volatile components is obtained in an amount of 17 ppm or less (preferably 0.1 ppm or more and 17 ppm or less) relative to the weight of the starting kelp.

Moreover, in the case of obtaining the volatile components with a molecular weight of less than 140 derived from kelp by distillation, the desired components can be obtained by collecting the solution captured from the start of the distillation of the solution containing the kelp soaked therein to the point at which the volatile components is obtained in an amount of 12 ppm or less (preferably 0.1 ppm or more and 12 ppm or less) relative to the weight of the starting kelp.

By discarding the second half of the solution collected in capturing the generated vapor, the volatile components with a molecular weight of 200 or more can be efficiently removed. For example, in the case where the solution is captured in fractions per 5% by weight, relative to the starting solvent, to give 40% by weight of fractions in total, eight capture containers are prepared and, when a 5% by weight portion, relative to the starting solvent, of the solution (hereinafter called the first liquid distillate) is obtained, the first capture container is quickly replaced by the second one to capture the next 5% by weight portion of the solution (the second liquid distillate). This procedure is repeated until the eighth liquid distillate is captured. In this case, the volatile components with a molecular weight of less than 200 contained in the solution are mainly contained in the first liquid distillate. From the second liquid distillate and subsequent ones, the content of the low-molecular weight components largely decreases while the content of the volatile components with a molecular weight of 200 or more gradually increases. In general, after starting the distillation, the ratio of the volatile components derived from kelp can be controlled to 20 ppm or less relative to the weight of the starting kelp and the volatile components with a molecular weight of less than 200 derived from kelp can be obtained by collecting the solution in an amount of 5 to 20% by weight relative to the starting solvent.

It is also possible to obtain the volatile components with a molecular weight of less than 200, i.e., the active ingredient of the invention, by capturing the solution in fractions while controlling the amount of the fractions so as to collect the solution until the volatile components derived from kelp is obtained in an amount of 20 ppm or less relative to the weight of the starting kelp.

As described above, the salty taste enhancer according to the invention contains the volatile components derived from kelp, preferably the volatile components with a molecular weight of less than 200 derived from kelp, more preferably the volatile components with a molecular weight of less than 170 derived from kelp, and still more preferably the volatile components with a molecular weight of less than 140 derived from kelp, as the active ingredient. For example, in such a salty taste enhancer which contains the volatile components with a molecular weight of less than 200 derived from kelp as the active ingredient, it is preferred to minimize the content of the volatile components with a molecular weight of 200 or more derived from kelp. However, it does not matter that the salty taste enhancer contains the volatile components with a molecular weight of 200 or more derived from kelp, so long as it can exert the salty taste-enhancing effect and the flavor enhancing effect and shows regulated characteristic flavors (the smell of the sea, algal smell and threadiness) inherent to kelp.

The salty taste enhancer of the invention can be obtained by collecting the solution until the volatile components is obtained in an amount of 20 ppm or less relative to the weight of the starting kelp and then stopping the distillation. However, in the case where the captured solution obtained by the distillation is mixed with non-volatile components of kelp to use as a kelp extract having a salty taste-enhancing effect as will be discussed hereinafter, it is preferred to further continue the distillation to thereby remove the entire volatile components from the starting kelp. Thus, the high-molecular weight volatile components contained in the starting kelp can be efficiently removed. Namely, after obtaining the volatile components in an amount of 20 ppm relative to the weight of the starting kelp, the captured solution is daringly collected and discarded. Then, the solution captured until the volatile components is obtained in an amount of 20 ppm or less relative to the weight of the starting kelp is mixed with the non-volatile components extracted. Thus, a high-quality kelp extract, which has a salty taste-enhancing effect without showing any smell of the sea, can be prepared.

Although methods for capturing the volatile components with the use of water as the solvent have been illustrated above in detail, it is also possible to use a dilute aqueous ethanol solution as the solvent to be used in the distillation for capturing the volatile components with a lower molecular weight derived from kelp. Also, it is possible to maintain the heating temperature at a relatively low level of 60 to 90° C. during the distillation. By using these methods, low-molecular weight volatile components can be captured at a higher ratio than the distillation merely using boiling water. However, when the captured components are to be mixed with the non-volatile components, as will be described hereinafter, to give a kelp extract having the salty taste-enhancing effect, these methods are somewhat disadvantageous. That is to say, in the former method, the amount of the non-volatile components extracted with the ethanol remaining in the kettle is reduced. In the latter method, on the other hand, the extraction of the non-volatile components requires a long time and, moreover, the undesirable high-molecular weight volatile components cannot be removed from the kelp extract. As a means for solving the problems in the latter method, use may be made of a procedure of elevating the kettle temperature at a gentle speed to thereby selectively collect the low-molecular weight volatile components, and then immediately elevating the kettle temperature to thereby rapidly collect the high-molecular weight volatile components.

Another method for obtaining the low-molecular weight volatile components from the captured solution obtained by the distillation comprises further subjecting the captured solution, which contains the volatile components, to solvent extraction, re-distillation, a chromatographic treatment and the like. The solvent extraction can be carried out by extracting the captured solution with the use of one or more kinds of solvents such as hexane, acetone, diethyl ether, pentane, ethanol, methanol and ethyl acetate via some steps, if necessary, to thereby selectively collect the low-molecular weight volatile components alone. The re-distillation can be carried out by treating the captured solution under reduced pressure to thereby selectively collect the low-molecular weight volatile components alone. The chromatographic treatment can be carried out by applying the captured solution to a column packed with an adsorbent such as active carbon, zeolite, silica gel, alumina, porous glass, an ion exchange resin, a synthetic adsorbent or cyclodextrin to thereby selectively collect the low-molecular weight volatile components alone. Further, two or more of these methods may be combined together to give a salty taste enhancer having improved effects.

As described above, the captured solution containing the volatile components derived from kelp, which is obtained by the distillation, may be directly used as a salty taste enhancer. Alternatively, the solvent may be removed from the captured solution so as to use the only volatile components derived from kelp, which are thus separated and concentrated, as a salty taste enhancer. The solvent may be removed form the captured solution by, for example, solvent extraction. Namely, components of the captured solution other than water are selectively extracted with a solvent such as hexane, acetone, diethyl ether, pentane, ethanol, methanol and ethyl acetate and the obtained extract is treated in vacuo to thereby remove the solvent.

The distillation methods have been described above in greater detail. Next, other methods for extracting the starting kelp, i.e., the organic solvent-extraction method and the supercritical extraction method will be illustrated.

In the organic solvent-extraction method, a solvent such as hexane, acetone, diethyl ether, pentane, ethanol, methanol and ethyl acetate is added to the starting kelp in a closed device. Then, the extraction is carried out at −20 to 90° C. for an hour to several days. In the extraction, it is preferred to control (the weight ratio of the kelp): (the solvent) to 1:100 to 1:5. After the completion of the extraction, the solvent is removed from the extract and the obtained concentrate is further applied to a multiplate distillation column and treated under reduced pressure. Thus, the volatile components of the invention can be obtained. In this method, the low-molecular weight volatile components, in particular, the volatile components with a molecular weight of less than 200 can be obtained by collecting the captured solution from the start of the distillation to the point at which the volatile components is obtained in an amount of 20 ppm or less (preferably 0.1 ppm or more and 20 ppm or less) relative to the weight of the starting kelp.

In the supercritical extraction method, the same procedure as in the organic solvent-extraction method is conducted in a closed device so that the volatile components and the volatile components with a molecular weight of less than 200 can be obtained.

It is also possible to combine two or more methods selected from these organic solvent-extraction method, supercritical extraction method and distillation method to give a salty taste enhancer having improved effects.

In the invention, the starting kelp may be subjected to a treatment with an enzyme (i.e., an enzymatic treatment) to capture preferable volatile components in a larger amount. Examples of the enzyme usable therefor include protease, glucosidase, glutaminase, nuclease, deaminase, cellulase, pectinase and the like. Either one or more kinds of enzymes may be used. The enzymatic treatment is performed before capturing the volatile components from the starting kelp. The enzymatic treatment is performed by, for example, feeding the kelp and water to a heating container, then adding an appropriate amount of the aforesaid enzyme thereto, heating the container to a temperature appropriate for the enzymatic treatment and, if necessary, stirring the mixture for an appropriate period of time. The enzymatic treatment may be conducted in a closed heating container equipped with a condenser tube for carrying out steam distillation and the like, and then the steam distillation, organic solvent-extraction, supercritical extraction, and the like may be carried out in the same device. Alternatively, the enzymatic treatment may be performed in another container and then the enzymatically treated kelp and solvent are transferred to a device for carrying out the steam distillation, organic solvent-extraction, supercritical extraction, and the like, followed by the steam distillation, organic solvent-extraction, supercritical extraction, and the like. In the case of transferring to the other container, it is preferred to cool to room temperature or lower so as to minimize the loss of the volatile components.

The enzyme is preferably used at a ratio of 0.01 to 10% by weight, more preferably 0.1 to 2% by weight, relative to the starting kelp. The temperature and period of the enzymatic treatment may depend on the optimum conditions of each enzyme without particular restriction. In general, it is preferred to carry out the enzymatic treatment at room temperature to 60° C. (more preferably 40 to 50° C.) for 10 minutes to 48 hours (more preferably 12 to 24 hours). After the completion of the enzymatic treatment, the enzyme should be inactivated by heating to 70 to 100° C. or adding an organic solvent. However, these procedures may be omitted, and the subsequent steps such as the steam distillation and solvent extraction may be quickly conducted. This method has some advantages, i.e., the same effect of inactivating the enzyme can be achieved while reducing the loss of the volatile components.

The salty taste enhancer containing the volatile components obtained by the aforesaid treatment can be directly used as a salty taste enhancer for foods or drinks. Alternatively, it may be distilled or concentrated to give a salty taste enhancer for foods or drinks. By distillation, a solution containing the volatile components, which can be used as a salty taste enhancer, can be directly obtained.

The salty taste enhancer of the invention may be dissolved in various liquids and used as a solution. Examples of the liquids include water, ethanol, edible fat or oil and the like. Any liquid suitable for edible use may be used without restriction.

The salty taste enhancer according to the invention may be used either as the volatile components derived from kelp per se or as a powder carrying a solution thereof enclosed therein. The latter product can be obtained by spray drying the volatile components, dissolving the same in the fat or oil, followed by the adsorption by dextrin, and the like.

The salty taste enhancer according to the invention may be mixed with non-volatile components (hereinafter also referred to as taste components) extracted from kelp to give a kelp extract having an enhanced salty taste. The non-volatile components of kelp may be extracted by subjecting the kelp, from which the volatile components derived from kelp are removed, to, for example, hot water-extraction, hot water-extraction combined with an enzymatic treatment, hot water-extraction under elevated pressure, hot water-extraction under elevated pressure combined with an enzymatic treatment, ethanol/water-extraction or the like. When water is used as the solvent in such a method of collecting the volatile components by distillation, for example, the solvent remaining in the heating container after the completion of the distillation corresponds to the above-described hot-water extract. It is preferred that the volatile components derived from kelp are completely removed from the extract obtained by extracting the non-volatile components from kelp. Since high-molecular weight volatile components, in particular, those having a molecular weight of 200 or more cause the smell of the sea and algal smell characteristic to kelp, the presence of these high-molecular weight volatile components, which scarcely vaporize, in the aforesaid extract brings about a reduction in preference and a narrowing in the application range of the kelp extract.

After extracting the non-volatile components, the extract is cooled in the case of being hot. Then, the salty taste enhancer of the invention, which contains the aforesaid volatile components as the active ingredient, is added thereto and uniformly mixed. Thus, an excellent kelp extract, which contains the volatile components having the salty taste-enhancing effect, can be obtained.

The extract obtained by, for example, separating the residual kelp, can be mixed with the salty taste enhancer of the invention by: (1) separating and removing the kelp residue from the kelp residue-containing extract, from which the volatile components derived from kelp are removed by distillation, to give an extract containing the non-volatile components, and then mixing it with the captured solution of the volatile components derived from kelp which are collected by the aforesaid distillation; or (2) cooling the kelp residue-containing extract, from which the volatile components derived from kelp are removed by distillation, then adding thereto a desired fraction of the above captured solution containing the volatile components derived from kelp which are collected, stirring the mixture for a definite period of time and then separating and removing the kelp residue therefrom. The method (1) is advantageous in that the loss of the aroma is low in the step of separating (filtering) the kelp residue, while the method (2) is advantageous in that a high handling efficiency can be achieved since the operations from extraction to mixing can be consistently performed in a single device.

With respect to the mixing ratio of the kelp extract and the salty taste enhancer, it is preferred to control the mixing ratio so that the non-volatile components derived from kelp is contained in the kelp extract in an amount of less than 100,000 times by weight relative to the volatile components derived from kelp contained in the salty taste enhancer. It is still preferred that the weight ratio of (the volatile components derived from kelp contained in the salty taste enhancer):(the non-volatile components derived from kelp in the kelp extract) is controlled to 1:5,000 to 1:50,000.

The kelp extract according to the invention having an enhanced salty taste contains volatile components with a molecular weight of less than 200, more preferably less than 170 and still more preferably less than 140. Also, the kelp extract preferably contains volatile components with a molecular weight of 200 or more derived from the starting kelp in an amount of less than 60% by weight, more preferably 0 or more and less than 50% by weight and still more preferably 0 or more and less than 40% by weight. In addition thereto, it is still preferred that the kelp extract contains volatile components with a molecular weight of 170 or more and less than 200 derived from the starting kelp in an amount of less than 80% by weight, more preferably 0 or more and less than 60% by weight and still more preferably 0 or more and less than 50% by weight. Moreover, it is preferred that the kelp extract contains volatile components with a molecular weight of 140 or more and less than 170 derived from the starting kelp in an amount of less than 75% by weight, more preferably 0 or more and less than 70% by weight.

The salty taste enhancer derived from kelp and kelp extract obtained in the invention can be mixed with other various seasoning materials for foods. Examples of the seasoning materials for foods include: inorganic salts such as sodium chloride, potassium chloride and magnesium sulfate; sugars and sugar alcohols such as sucrose, glucose, fructose, maltose, oligosaccharides, dextrin, starch, sorbitol, xylitol, maltol and reduced sugar syrup; natural seasonings such as soy sauce, miso (fermented bean paste), cheese, spice, vinegar, tomato ketchup, Worcestershire sauce, meat extracts, fish extracts, vegetable extracts, spices, protein hydrolysates, protease-digested matters and yeast extracts; taste seasonings such as sodium glutamate, sodium guanylate and sodium inosinate; acidic ingredients such as citric acid, succinic acid and lactic acid; other flavors or fragrances, spice extracts, antioxidants, pH-adjusting agents, smoke solutions and the like.

The salty taste enhancer derived from kelp obtained in the present invention and the kelp extract comprising the same can be used together with other salty taste-enhancing components the salty taste-enhancing effects of which have been reported.

Examples of the other salty taste-enhancing components include potassium chloride, acidic ingredients, yeast extracts and on the like. For example, when the salty taste enhancer of the invention is combined with potassium chloride, not only the effect of relieving the harshness of potassium chloride having been reported so far but an effect of enhancing the salty taste-like top impact, which cannot be achieved by the existing salty taste-enhancing components and the like, can be obtained. In this case, therefore, an improved salty taste-enhancing effect can be expected.

Also, the salty taste enhancer derived from kelp and kelp extract of the invention can be used in various foods or drinks. Examples of the foods or drinks in which the salty taste enhancer derived from kelp or kelp extract of the invention is usable include various kinds of foods or drinks, for example, snack foods; confectionaries such as candies, chewing gums and biscuits; frozen desserts such as ice creams and sherbets; foods for microwave cooking; retort foods such as curries, sauces for rice bowl dishes and pasta sauce; soups; meat/fish paste products; dressings and mayonnaise; taste seasonings; dips; liquid seasonings such as noodle soups and ponzu (citrus-flavored vinegar); instant noodles; powdery seasonings such as table salt, furikake (dried seasoning powders), ochaduke no moto (dried seasoning powders to be taken with rice and green tea) and seasonings for pasta; drinks such as soft drinks, fruit juices and carbonated drinks; jams and fruit preserves; western confectioneries such as cakes, bavarois and mousses; cooked products; prepared foods; food delicacies; baked products; margarines and on the like.

The salty taste enhancer derived from kelp or kelp extract of the invention may be used in various foods or drinks at an arbitrary ratio. For example, they may be added in such a manner as to control the ratio of the volatile components derived from kelp to 0.01 ppb to 1 ppm, preferably 0.05 to 100 ppb and more preferably 0.1 to 50 ppb, relative to the weight of the food or drink.

### EXAMPLES

To further illustrate the invention in greater detail, the following Examples will be given. However, it is to be understood that the invention is not restricted thereto. In the following Examples, the term “room temperature” means a temperature of 20 to 30° C.

### Example 1

Into a tightly sealed heating kettle equipped with a condenser tube, 500 g of water and 50 g of dry kelp (Laminaria angustata: manufactured by Maruhachi Muramatsu Corporation) were fed and heated. After the temperature attained 100° C., the heating was continued for 120 minutes under stirring. The vapor thus generated was captured through the condenser tube. Thus, 150 g of a liquid distillate, which was referred to as kelp aroma P, was captured. The content of volatile components derived from kelp in the liquid distillate was 30.0 ppm relative to the starting kelp.

After cooling the extract part in the kettle to room temperature, kelp aroma P having been captured was returned into the kettle and the mixture was stirred for 10 minutes. After removing the extraction residue, 350 g of kelp extract A was obtained.

### Comparative Example 1

Into a non-sealed heating kettle, 500 g of water and 50 g of dry kelp (Laminaria angustata: manufactured by Maruhachi Muramatsu Corporation) were fed and heated. After the temperature attained 100° C., the heating was continued for 120 minutes under stirring. After cooling to room temperature, the weight loss caused by vaporization during the heating was weighed. As a result, the weight loss was 150 g. After adding 150 g of water, the mixture was stirred for 10 minutes and then the extraction residue was removed. Thus, 350 g of kelp extract B was obtained.

### Example 2

Into a tightly sealed heating kettle equipped with a condenser tube, 500 g of water, 50 g of dry kelp (Laminaria angustata: manufactured by Maruhachi Muramatsu Corporation), 0.5 g of protease (Protease A “Amano” G; manufactured by Amano Enzyme Inc.), 0.3 g of cellulase (Cellulosin AC40; manufactured by HBI Enzymes Inc.) and 0.2 g of glutaminase (Glutaminase Daiwa SD-C100S; manufactured by Amano Enzyme Inc.) were fed and heated under stirring. The kelp was enzymatically treated by maintaining at 40° C. for 16 hours. Next, the temperature was elevated to 100° C. and the heating was continued for 120 minutes. The vapor thus generated was captured through the condenser tube. The first 25 g portion was referred to as liquid distillate L, the second 25 g portion was referred to as liquid distillate M, the third 50 g portion was referred to as liquid distillate N, and the final 50 g portion was referred to as liquid distillate O. The mixture of liquid distillates L to O was referred to as kelp aroma Q. After cooling the extract part in the kettle to room temperature, kelp aroma Q was added to the kettle and the mixture was stirred for 10 minutes. After removing the extraction residue, 350 g of kelp extract C was obtained.

### Test Example 1

### Aroma Analysis 1

Liquid distillates L, M, N and O obtained above were pre-treated in the following manner and then analyzed by GC (gas chromatography)/FID (flame ionization detector) and GC/MS (mass spectrometry) to analyze the aromas. Table 1 shows the contents of typical components, expressed by referring an internal standard as to 1, and the molecular weights thereof. Gas chromatography was conducted under the following conditions.

### [Conditions for Pre-Treating Samples]

(a) By adding 200 g of refined salt, 400 g of a liquid distillate was salted-out. As an internal standard, 100 μl of a nonan-5-one diethyl ether solution (100 mg/100 ml) was added and dissolved.

(b) The mixture was subjected to liquid/liquid extraction with diethyl ether using a separating funnel (100 ml×2).

(c) The mixture was dehydrated by adding 10 to 20 g of anhydrous sodium sulfate.

(d) The dehydrated mixture was concentrated to about 100 mg by distilling off the solvent to give a sample for GC analysis.

### [Gas Chromatography Conditions]

### (Conditions)

Gas chromatography for GC/FID analysis: HP6890N (manufactured by Agilent Technologies)

Gas chromatography for GC/MS analysis: GCMS-QP2010 (manufactured by Shimadzu Corporation)

Column: Rxi-5-ms (50 m×0.25 mm, df=0.15 μm) (manufactured by Restech)

Temperature program: 50° C. (1 min)→230° C. (rising at 4° C./min)→294° C. (rising at 8° C./min)

TABLE 1

| | | Liquid | Liquid | Liquid | Liquid |
|---|---|---|---|---|---|
| | Molecular | distil- | distil- | distil- | distil- |
| Component | weight | late L | late M | late N | late O |
| Hexanal | 100 | 3.43 | 0.23 | 0.11 | 0.06 |
| Heptanal | 114 | 0.59 | 0.06 | N.D. | N.D. |
| 1-Octen-3-ol | 128 | 10.11 | 0.47 | 0.57 | 0.33 |
| 2E-octenol | 128 | 5.71 | 0.34 | 0.34 | N.D. |
| 2E-nonenal | 140 | 1.55 | 0.15 | 0.27 | 0.21 |
| 2E,4E-decadienal | 152 | 0.72 | 0.08 | 0.16 | N.D. |
| 3,5-Octadien-3-one | 166 | 0.77 | 0.09 | 0.17 | N.D. |
| β-Ionone | 192 | 1.88 | 0.25 | 0.51 | 0.43 |
| Myristic acid | 228 | 17.19 | 2.17 | 5.63 | 10.89 |
| Palmitic acid | 256 | 3.70 | 0.45 | 1.42 | 5.04 |

N.D.: not detected (less than detection limit)

### Test Example 2

### Aroma Analysis 2)

A mixture of liquid distillates (L, M, N and O), a mixture of liquid distillates (L, M and N), a mixture of liquid distillates (L and M) and liquid distillate (L), each obtained in Example 2, were subjected to the same aroma analysis as in Test Example 1. Table 2 shows the results. The amounts of the samples employed are as follows: the mixture of liquid distillates (L, M, N and O): 150 g (liquid distillate L: 25 g, liquid distillate M: 25 g, liquid distillate N: 50 g, liquid distillate O: 50 g), the mixture of liquid distillates (L, M and N): 100 g (liquid distillate L: 25 g, liquid distillate M: 25 g, liquid distillate N: 50 g), the mixture of liquid distillates (L and M): 50 g (liquid distillate L: 25 g, liquid distillate M: 25 g), and liquid distillate (L): 25 g. The internal standard solution was used in an amount of 6.0 μl. Liquid distillates L to O contained all of the volatile components of the starting kelp.

TABLE 2

| | | | Liquid distillates | | Liquid distillates | | Liquid distillate | |
|---|---|---|---|---|---|---|---|---|
| | | | (L, M & N) | | (L & M) | | (L) | |
| | Molecular | Liquid distillates | | Percentage to | | Percentage to | | Percentage to |
| Component | weight | (L, M, N & O) | | (L, M, N & O) | | (L, M, N & O) | | (L, M, N & O) |
| Hexanal | 100 | 4.00 | 3.88 | 97 | 3.66 | 91 | 3.43 | 86 |
| Heptanal | 114 | 0.65 | 0.65 | 100 | 0.65 | 100 | 0.59 | 91 |
| 1-Octen-3-ol | 128 | 12.38 | 11.73 | 95 | 10.58 | 85 | 10.11 | 82 |
| 2E-octenol | 128 | 6.73 | 6.73 | 100 | 6.04 | 90 | 5.71 | 85 |
| 2E-nonenal | 140 | 2.65 | 2.24 | 85 | 1.70 | 64 | 1.55 | 58 |
| 2E,4E-decadienal | 152 | 1.12 | 1.12 | 100 | 0.80 | 71 | 0.72 | 64 |
| 3,5-Octadien-3-one | 166 | 1.21 | 1.21 | 100 | 0.86 | 71 | 0.77 | 64 |
| β-Ionone | 192 | 3.98 | 3.12 | 78 | 2.10 | 53 | 1.85 | 46 |
| Myristic acid | 228 | 52.41 | 30.63 | 58 | 19.37 | 37 | 17.19 | 33 |
| Palmitic acid | 256 | 17.07 | 6.98 | 41 | 4.15 | 24 | 3.70 | 22 |

The content of the volatile components in liquid distillate (L) was 11.0 ppm relative to the weight of the starting kelp, the content of the volatile components in liquid distillates (L and M) was 17.6 ppm relative to the weight of the starting kelp, the content of the volatile components in liquid distillates (L, M and N) was 21.6 ppm relative to the weight of the starting kelp, and the content of the volatile components in liquid distillates (L, M, N and O) was 35.4 ppm relative to the weight of the starting kelp.

### Example 3

25 g of liquid distillate L, 25 g of liquid distillate M, 50 g of liquid distillate N and 50 g of liquid distillate O were obtained in the same manner of Example 2. A mixture of liquid distillates L, M and N and 50 g of water was referred to as kelp aroma R.

### Example 4

25 g of liquid distillate L, 25 g of liquid distillate M, 50 g of liquid distillate N and 50 g of liquid distillate O were obtained in the same manner of Example 2. A mixture of liquid distillates L and M with 100 g of water was referred to as kelp aroma S. After cooling the extract part in the kettle to room temperature, kelp aroma S was added to the kettle and the mixture was stirred for 10 minutes. After removing the extraction residue, 350 g of kelp extract D was obtained.

### Example 5

25 g of liquid distillate L, 25 g of liquid distillate M, 50 g of liquid distillate N and 50 g of liquid distillate O were obtained in the same manner of Example 2. A mixture of liquid distillate L with 125 g of water was referred to as kelp aroma T. After cooling the extract part in the kettle to room temperature, kelp aroma T was added to the kettle and the mixture was stirred for 10 minutes. After removing the extraction residue, 350 g of kelp extract E was obtained.

The kelp extract obtained in Example 1 contained 38% by weight of non-volatile components relative to the starting kelp. The kelp extracts obtained in Examples 2, 4 and 5 each contained 42% by weight of non-volatile components relative to the starting kelp.

### Comparative Example 2

Into a non-sealed heating kettle, 500 g of water, 50 g of dry kelp (Laminaria angustata: manufactured by Maruhachi Muramatsu Corporation), 0.5 g of protease (Protease A “Amano” G; manufactured by Amano Enzyme Inc.), 0.3 g of cellulase (Cellulosin AC40; manufactured by HBI Enzymes Inc.) and 0.2 g of glutaminase (Glutaminase Daiwa SD-C100S; manufactured by Amano Enzyme Inc.) were fed and heated under stirring. The kelp was enzymatically treated by maintaining at 40° C. for 16 hours. Next, the temperature was elevated to 100° C. and the heating was continued for 120 minutes. After cooling to room temperature, the weight loss caused by vaporization during the heating was weighed. As a result, the weight loss was 150 g. After adding 150 g of water, the mixture was stirred for 10 minutes and then the extraction residue was removed. Thus, 350 g of kelp extract F was obtained.

### Comparative Example 3

Into a tightly sealed heating kettle equipped with a condenser tube, 500 g of water and 50 g of dry kelp (Laminaria angustata: manufactured by Maruhachi Muramatsu Corporation) were fed and heated. After the temperature attained 100° C., the heating was continued for 120 minutes under stirring. The vapor thus generated was captured through the condenser tube. Thus, 150 g of a liquid distillate was captured. After cooling the extract part in the kettle to room temperature, 150 g of water was added. After removing the extraction residue, 350 g of kelp extract G was obtained.

### Comparative Example 4

Into a tightly sealed heating kettle equipped with a condenser tube, 500 g of water, 50 g of dry kelp (Laminaria angustata: manufactured by Maruhachi Muramatsu Corporation), 0.5 g of protease (Protease A “Amano” G; manufactured by Amano Enzyme Inc.), 0.3 g of cellulase (Cellulosin AC40; manufactured by HBI Enzymes Inc.) and 0.2 g of glutaminase (Glutaminase Daiwa SD-C100S; manufactured by Amano Enzyme Inc.) were fed and heated under stirring. The kelp was enzymatically treated by maintaining at 40° C. for 16 hours. Next, the temperature was elevated to 100° C. and the heating was continued for 120 minutes. The vapor thus generated was captured through the condenser tube. The first 25 g portion was referred to as liquid distillate L, the second 25 g portion was referred to as liquid distillate M, the third 50 g portion was referred to as liquid distillate N and the final 50 g portion was referred to as liquid distillate O.

After cooling the extract part in the kettle to room temperature, 150 g of water was added. After removing the extraction residue, 350 g of kelp extract H was obtained.

### Test Example 3

### Sensory Evaluation

By 9 skilled panelists, foods containing kelp extracts A to H, kelp aromas P and T and liquid distillates M to O, that were obtained in Examples 1 to 5 and Comparative Examples 1 to 4, were taken and sensorily evaluated. The sensory evaluations were conducted by the following methods.

### Sensory Evaluation 1

### Preparation of Noodle Soup to be Evaluated

In accordance with the ratios listed in Tables 3 and 4, materials were mixed at room temperature and enclosed in a sealed container. Then the samples were heated in hot water until the temperature attained 90° C. to give 3-fold concentrated noodle soup samples of controls 1 and 2, Evaluation Examples 1 to 7 and Comparative Evaluation Examples 1 to 8. Control 1 was 3-fold concentrated noodle soups commonly used. Each noodle soup was mixed with 5 times as much as water and stirred. 100 ml of the mixture was poured into a 200 ml beaker to give a sample to be evaluated.

### Evaluation Method

The evaluation was made in 4 items including “deliciousness”, “total flavor strength”, “top impact of flavor” and “salty taste”. By referring control 1 as to 5 points, the preference in each item was scored on a scale of 1 to 7 (full) points. The results were expressed in the average of 9 panelists. Tables 3 and 4 show the results.

TABLE 3

| | | | Comp. | Comp. | | | | | Comp. | Comp. | Comp. | |
|---|---|---|---|---|---|---|---|---|---|---|---|---|
| Starting | | | Eval- | Eval- | Eval- | Eval- | Eval- | Eval- | Eval- | Eval- | Eval- | Eval- |
| material | | | uation | uation | uation | uation | uation | uation | uation | uation | uation | uation |
| (salt | Control | Control | Exam- | Exam- | Exam- | Exam- | Exam- | Exam- | Exam- | Exam- | Exam- | Exam- |
| content: %) | 1 | 2 | ple 1 | ple 2 | ple 1 | ple 2 | ple 3 | ple 4 | ple 3 | ple 4 | ple 5 | ple 5 |
| Soy sauce | 20.0 | 20.0 | 20.0 | 20.0 | 20.0 | 20.0 | 20.0 | 20.0 | 20.0 | 20.0 | 20.0 | 20.0 |
| (13.6%) | | | | | | | | | | | | |
| Sugar | 15.0 | 15.0 | 15.0 | 15.0 | 15.0 | 15.0 | 15.0 | 15.0 | 15.0 | 15.0 | 15.0 | 15.0 |
| Sodium | 4.7 | 2.1 | 2.1 | 2.1 | 2.1 | 2.1 | 2.1 | 2.1 | 2.1 | 2.1 | 2.1 | 2.1 |
| chloride | | | | | | | | | | | | |
| Bonito extract | 10.0 | 10.0 | 10.0 | 10.0 | 10.0 | 10.0 | 10.0 | 10.0 | 10.0 | 10.0 | 10.0 | 10.0 |
| (12%) | | | | | | | | | | | | |
| Potassium | — | — | — | — | — | — | — | — | — | — | 1.5 | 1.5 |
| chloride | | | | | | | | | | | | |
| Kelp extract of | — | — | — | — | A: 0.1 | C: 0.1 | D: 0.1 | E: 0.1 | — | — | — | E: 0.1 |
| the invention | | | | | | | | | | | | |
| Kelp extract | — | — | B: 0.1 | F: 0.1 | — | — | — | — | G: 0.1 | H: 0.1 | — | — |
| Total (adjusted | 100.0 | 100.0 | 100.0 | 100.0 | 100.0 | 100.0 | 100.0 | 100.0 | 100.0 | 100.0 | 100.0 | 100.0 |
| with water) | | | | | | | | | | | | |
| Salt content | 8.6% | 6.0% | 6.0% | 6.0% | 6.0% | 6.0% | 6.0% | 6.0% | 6.0% | 6.0% | 6.0% | 6.0% |
| Deliciousness | 5.0 | 2.6 | 2.9 | 3.0 | 3.4 | 3.6 | 4.1 | 4.3 | 2.9 | 3.0 | 3.0 | 4.5 |
| Total flavor | 5.0 | 2.0 | 2.9 | 3.0 | 3.6 | 4.0 | 4.1 | 4.1 | 2.9 | 3.0 | 3.6 | 5.0 |
| strength | | | | | | | | | | | | |
| Top impact of | 5.0 | 1.9 | 2.4 | 2.4 | 3.3 | 3.7 | 4.0 | 4.3 | 2.4 | 2.1 | 3.0 | 4.9 |
| flavor | | | | | | | | | | | | |
| Salty taste | 5.0 | 1.5 | 2.0 | 2.1 | 2.8 | 3.0 | 3.3 | 3.4 | 2.0 | 2.1 | 3.4 | 4.1 |

The amounts of starting materials are expressed in % by weight.

TABLE 4

| | | | | | Comp. | Comp. | Comp. |
|---|---|---|---|---|---|---|---|
| Starting material | Control | Control | Evaluation | Evaluation | Evaluation | Evaluation | Evaluation |
| (salt content: %) | 1 | 2 | Example 6 | Example 7 | Example 6 | Example 7 | Example 8 |
| Soy sauce (13.6%) | 20.0 | 20.0 | 20.0 | 20.0 | 20.0 | 20.0 | 20.0 |
| Sugar | 15.0 | 15.0 | 15.0 | 15.0 | 15.0 | 15.0 | 15.0 |
| Sodium chloride | 4.7 | 2.1 | 2.1 | 2.1 | 2.1 | 2.1 | 2.1 |
| Bonito extract (12%) | 10.0 | 10.0 | 10.0 | 10.0 | 10.0 | 10.0 | 10.0 |
| Potassium chloride | — | — | — | — | — | — | — |
| Salty taste enhancer | — | — | Kelp | Kelp | Liquid | Liquid | Liquid |
| | | | aroma P | aroma T | distillate M | distillate N | distillate O |
| | | | 0.025 | 0.025 | 0.025 | 0.025 | 0.025 |
| Total (adjusted with water) | 100.0 | 100.0 | 100.0 | 100.0 | 100.0 | 100.0 | 100.0 |
| Salt content | 8.6% | 6.0% | 6.0% | 6.0% | 6.0% | 6.0% | 6.0% |
| Deliciousness | 5.0 | 2.6 | 3.8 | 4.5 | 2.7 | 2.4 | 1.9 |
| Total flavor strength | 5.0 | 2.0 | 3.5 | 3.8 | 2.2 | 2.4 | 2.5 |
| Top impact of flavor | 5.0 | 1.9 | 3.6 | 4.4 | 2.2 | 2.0 | 1.8 |
| Salty taste | 5.0 | 1.5 | 3.0 | 3.8 | 1.8 | 1.7 | 1.5 |

The amounts of starting materials are expressed in % by weight.
“Liquid distillate M” in the table was prepared by adding water to 25 g of liquid distillate M to give a total amount of 150 g.
“Liquid distillate N” in the table was prepared by adding water to 50 g of liquid distillate N to give a total amount of 150 g.
“Liquid distillate O” in the table was prepared by adding water to 50 g of liquid distillate O to give a total amount of 150 g.

Tables 3 and 4 indicate the following facts.

(1) Control 2 had a salt content smaller by about 30% by weight than control 1. Due to this salt reduction, the total flavor of control 2 was considerably worsened compared with control 1. In particular, control 2 showed low evaluation scores in salty taste and top impact.

(2) Although the sample of Comparative Evaluation Example 1 contained no salty taste enhancer of the invention, it showed an effect of somewhat enhancing salty taste, etc., due to the kelp extract added thereto, compared with control 2. However, Comparative Evaluation Example 1 was not highly evaluated in deliciousness and top impact.

(3) Although the sample of Comparative Evaluation Example 2 contained no salty taste enhancer of the invention, it showed a somewhat strong taste and highly evaluated in deliciousness compared with Comparative Evaluation Example 1, because of containing, as kelp extract, the enzymatically treated kelp extract. However, no large difference was observed in other items between Comparative Evaluation Examples 1 and 2.

(4) In Evaluation Example 1, kelp extract containing all of the volatile components derived from kelp, i.e., the salty taste enhancer according to the invention, was used. Thus, the sample of Evaluation Example 1 had largely improved total flavor and top impact as well as enhanced salty taste, etc. compared with Comparative Evaluation Example 1. From the standpoint of flavor, it somewhat showed the smell of the sea characteristic to kelp.

(5) The sample of Evaluation Example 2 showed higher scores in total than the sample of Evaluation Example 1.

(6) The sample of Evaluation Example 3 showed enhanced top impact and regulated smell of the sea, compared with Evaluation Example 2, and also showed increase in the deliciousness and salty taste scores.

(7) In Evaluation Example 4, the effects of Evaluation Example 3 were further enhanced.

(8) Although the sample of Evaluation Example 6 somewhat showed the smell of the sea, it showed higher scores in the flavor with saltiness and top impact than Comparative Evaluation Example 1.

(9) The sample of Evaluation Example 7 showed much less smell of the sea, strengthened top impact and highly scored salty taste and deliciousness, compared with Evaluation Example 6.

(10) The sample of Comparative Evaluation Example 5 showed considerably strengthened salty taste and total flavor compared with Comparative Evaluation Example 1. However, it also showed strong harshness and, therefore, similarly evaluated in deliciousness as Comparative Evaluation Example 1.

(11) The sample of Evaluation Example 5 showed relieved harshness, compared with Comparative Evaluation Example 5, and largely enhanced total flavor and top impact. Thus, it was highly evaluated in salty taste and deliciousness.

(12) The sample of Comparative Evaluation Example 6 showed somewhat stronger flavor than control 2 but exerted only little effect of enhancing salty taste or top impact. It was also less preferred due to smell of the sea.

(13) The sample of Comparative Evaluation Example 7 was almost similar to the sample of Comparative Evaluation Example 6 but was somewhat less preferred because of having stronger smell of the sea.

(14) The sample of Comparative Evaluation Example 8 showed strong smell of the sea without any effect of enhancing salty taste or top impact.

### Sensory Evaluation 2

### Potato Chips

8 parts by weight of kelp extract E obtained in Example 5 was mixed with 92 parts by weight of dextrin (Pinedexd No. 100; manufactured by Matsutani Chemical Industry Co., Ltd.) and then the mixture was spray-dried to give a kelp extract powder having a salty taste-enhancing effect. 0.3% by weight of this kelp extract powder was added to commercially available potato chips having a salt content controlled to 1.0% by weight.

For comparison, 0.3% by weight of the aforesaid dextrin was added, as a substitute for the kelp extract powder according to the invention, to commercially available potato chips.

The potato chips using the kelp extract powder according to the invention showed an effect of significantly enhancing salty taste, compared with the comparative product.

### Sensory Evaluation 3

### Cup Noodles

50 parts by weight of liquid distillate L obtained in Example 5 and 50 parts by weight of salad oil were stirred in a closed container equipped with rotational blades for 1 hour at 40° C. After allowing to stand overnight, the oily phase was taken out and residual water was removed from the oily phase alone using sodium sulfate to give a kelp-flavored oil. To commercially available cup noodles, 0.1% weight of this kelp-flavored oil was added before eating. Similarly, 0.1% by weight of salad oil was added, as a substitute for the kelp-flavored oil for comparison.

The kelp-flavored oil using the salty taste enhancer according to the invention showed an effect of significantly enhancing salty taste, compared with the comparative product.

### Test Example 4

### Salt Content Analysis

The sodium contents of kelp extracts A to F, which were obtained in Examples 1, 2, 4 and 5 and Comparative Examples 1 and 2, were measured by the atomic absorption spectrometry (flame method). As a result, all of these kelp extracts contained about 170 mg/100 g of sodium.

### Example 6

Into a tightly sealed heating kettle, 500 g of water, 50 g of dry kelp (Laminaria angustata: manufactured by Maruhachi Muramatsu Corporation), 0.5 g of protease (Protease A “Amano” G; manufactured by Amano Enzyme Inc.), 0.3 g of cellulase (Cellulosin AC40; manufactured by HBI Enzymes Inc.) and 0.2 g of glutaminase (Glutaminase Daiwa SD-C100S; manufactured by Amano Enzyme Inc.) were fed and heated under stirring. The kelp was enzymatically treated by maintaining at 40° C. for 16 hours. Next, the mixture was cooled to room temperature and then 500 g of diethyl ether was added thereto. After mixing by stirring, the mixture was filtered to give a liquid mixture of water/diethyl ether. This liquid mixture was allowed to stand in a closed container for 2 hours to thereby divide into an aqueous phase and a diethyl ether phase. The diethyl ether phase alone was separated and collected. To the diethyl ether phase thus obtained, 500 g of additional diethyl ether was added and the same separation operation was conducted. Thus, a diethyl ether solution from which the aqueous phase had been removed was obtained. From this solution, diethyl ether was removed under reduced pressure. Thus, about 3 mg of a high-concentration organic solvent-extract of kelp A was obtained.

### Example 7

About 3 mg of high-concentration organic solvent-extract of kelp A, which was obtained in the same manner as in Example 6, was further distilled using a multiplate distillation column under reduced pressure. About 0.8 mg of volatile components obtained first were referred to as organic solvent-extract of kelp B (salty taste enhancer according to the invention).

### Example 8

Organic solvent-extracts of kelp A and B obtained in Examples 6 and 7 were each diluted with water to give a total amount of 150 g. Using the same procedures as described in Evaluation Example 6 in Sensory Evaluation 1 of Test Example 3, noodle soups were prepared and sensorily evaluated. As a result, the noodle soup using organic solvent-extract of kelp A showed an effect of highly enhancing salty taste comparable to the product of Evaluation Example 6. On the other hand, the noodle soup using organic solvent-extract of kelp B showed an effect of highly enhancing salty taste comparable to the product of Evaluation Example 7.

While the invention has been described in detail and with reference to specific embodiments thereof, it will be apparent to one skilled in the art that various changes and modifications can be made therein without departing from the spirit and scope thereof. This application is based on Japanese Patent Application No. 2010-089332 filed on Apr. 8, 2010 and Japanese Patent Application No. 2011-082883 filed on Apr. 4, 2011, the entire subject matter of which is incorporated herein by reference.

### INDUSTRIAL APPLICABILITY

The salty taste enhancer according to the invention can enhance salty taste without giving any odd-after tastes such as harshness as shown by potassium chloride, exert a flavor enhancing effect, which is another effect of sodium chloride in foods or drinks, on foods or drinks, and impart top impact, which is lost by salt reduction, to a food or drink.

In the salty taste enhancer according to the invention, furthermore, the characteristic flavors (the smell of the sea, algal smell and threadiness) inherent to kelp are relieved. Therefore, it can exert the salty taste-enhancing effect and flavor enhancing effect on foods or drinks over a wide range without giving any unpleasant feeling.

1. A salty taste enhancer comprising a volatile component with a molecular weight of less than 200 derived from a kelp.

2. The salty taste enhancer according to claim 1, wherein the kelp is an enzymatically treated kelp.

3. The salty taste enhancer according to claim 1, which comprises a volatile component with a molecular weight of 200 or more derived from the kelp in an amount of less than 60% by weight relative to an amount of a volatile component with a molecular weight of 200 or more contained in the starting kelp.

4. A method for producing the salty taste enhancer according to claim 1, which comprises distilling a kelp to give a salty taste enhancer comprising a volatile component with a molecular weight of less than 200 derived from the kelp.

5. A method for producing the salty taste enhancer according to claim 3, which comprises distilling a kelp to give a salty taste enhancer comprising: a volatile component with a molecular weight of less than 200 derived from the kelp; and a volatile component with a molecular weight of 200 or more derived from the kelp in an amount of less than 60% by weight relative to an amount of a volatile component with a molecular weight of 200 or more contained in the starting kelp.

6. The method according to claim 5, wherein a volatile component is captured from the start of the distillation to a point at which the volatile component is obtained in an amount of 20 ppm or less relative to the weight of the starting kelp.

7. The method according to claim 4, wherein the distillation is conducted after the kelp was treated with an enzyme.

8. A kelp extract which comprises a mixture of the salty taste enhancer according to claim 1 and an extract containing a non-volatile component of the kelp.

9. The kelp extract according to claim 8, wherein the extract containing a non-volatile component of the kelp is an extract containing a non-volatile component of the kelp, which is obtained after distilling the kelp to remove a volatile component from the kelp.

10. A food or drink comprising the salty taste enhancer according to a claim 1.

11. (canceled)

12. The salty taste enhancer according to claim 2, which comprises a volatile component with a molecular weight of 200 or more derived from the kelp in an amount of less than 60% by weight relative to an amount of a volatile component with a molecular weight of 200 or more contained in the starting kelp.

13. A method for producing the salty taste enhancer according to claim 12, which comprises distilling a kelp to give a salty taste enhancer comprising: a volatile component with a molecular weight of less than 200 derived from the kelp; and a volatile component with a molecular weight of 200 or more derived from the kelp in an amount of less than 60% by weight relative to an amount of a volatile component with a molecular weight of 200 or more contained in the starting kelp.

14. The method according to claim 13, wherein a volatile component is captured from the start of the distillation to a point at which the volatile component is obtained in an amount of 20 ppm or less relative to the weight of the starting kelp.

15. A kelp extract which comprises a mixture of the salty taste enhancer according to claim 2 and an extract containing a non-volatile component of the kelp.

16. A kelp extract which comprises a mixture of the salty taste enhancer according to claim 3 and an extract containing a non-volatile component of the kelp.

17. A kelp extract which comprises a mixture of the salty taste enhancer according to claim 12 and an extract containing a non-volatile component of the kelp.

18. The kelp extract according to claim 15, wherein the extract containing a non-volatile component of the kelp is an extract containing a non-volatile component of the kelp, which is obtained after distilling the kelp to remove a volatile component from the kelp.

19. The kelp extract according to claim 16, wherein the extract containing a non-volatile component of the kelp is an extract containing a non-volatile component of the kelp, which is obtained after distilling the kelp to remove a volatile component from the kelp.

20. The kelp extract according to claim 17, wherein the extract containing a non-volatile component of the kelp is an extract containing a non-volatile component of the kelp, which is obtained after distilling the kelp to remove a volatile component from the kelp.

21. A food or drink comprising the salty taste enhancer according to claim 2.

22. A food or drink comprising the salty taste enhancer according to claim 3.

23. A food or drink comprising the salty taste enhancer according to claim 12.
